(19) United States
(12) Patent Application Publication
(10) Pub. No.: US 2023/0145161 A1
(43) Pub. Date: May 11, 2023

(54) INFORMATION PROCESSING DEVICE, ENTRY SCREEN DISPLAY METHOD, AND MEDICAL SYSTEM

(71) Applicant: OLYMPUS CORPORATION, Tokyo (JP)

(72) Inventors: Kazuyoshi TAMURA, Tokyo (JP); Atsushi KODA, Tokyo (JP); Tomonori SATO, Tokyo (JP); Hideki YASUDA, Tokyo (JP)

(73) Assignee: OLYMPUS CORPORATION, Tokyo (JP)

(21) Appl. No.: 18/096,611

(22) Filed: Jan. 13, 2023

Related U.S. Application Data
(63) Continuation of application No. PCT/JP2020/027237, filed on Jul. 13, 2020.

(51) Int. Cl. G16H 10/60; G16H 10/20

(52) U.S. Cl. CPC G16H 10/60; G16H 10/20

(57) ABSTRACT

An order information recording unit records information on an order for a patient. An arrival state recording unit records an arrival state that indicates whether or not a patient has arrived, in association with an order for the patient. An accepting unit accepts patient identification information entered into a portable terminal device. An order retrieval unit retrieves an order related to the patient identification information accepted by the accepting unit. A decision processing unit decides to display an entry screen for medical interview information when the arrival state associated with an order related to the patient identification information indicates that the patient has arrived. A display processing unit displays the entry screen for the medical interview information on the portable terminal device, in response to the decision made by the decision processing unit.

### CROSS-REFERENCE TO RELATED APPLICATIONS

This application is based upon and claims the benefit of priority from the International Application No. PCT/JP2020/027237, filed on Jul. 13, 2020, the entire contents of which are incorporated herein by reference.

### BACKGROUND

### 1. Field of the Disclosure

The present disclosure relates to a technology for supporting appropriate entries of medical interview information.

### 2. Description of the Related Art

Patent Literature 1 discloses a medical information management apparatus that extracts, from implementation information on medical practice performed on a patient, information necessary to create an interview result for an interview item of the patient and creates the interview result. With the medical information management apparatus, interview results can be created more easily compared to the case where a nurse manually enters, into a database, answers to interview items filled by a patient on paper.
Patent Literature 1: Japanese Unexamined Patent Application Publication No. 2007-328740

Medical facilities have conventionally given patients a paper medical questionnaire and a pen to ask them to fill in answers (medical interview information) to questions (interview items) prior to medical practice. In recent years, however, it has become more common to have patients enter their medical interview information on tablets or other portable terminal devices. With the medical interview information entered into a tablet, while the entered medical interview information can be used as data, there arises a need for appropriate data management.

The medical interview information entered by a patient into a tablet needs to be accurately related to the patient's order. In conventional operation, a nurse displays an order list of the day on a tablet to select an examination order for a patient and performs various operations to display a medical interview information entry screen for the patient. The entry screen thus displayed is then related to the patient's order, and the nurse hands to the patient the tablet displaying the medical interview information entry screen. According to this operation, if the nurse selects a wrong order, the medical interview information entered on the entry screen will be related to an order different from the order to which the medical interview information should be actually related. Therefore, it is desired to develop a mechanism for reducing such human errors.

### SUMMARY

The present disclosure has been made in view of such a situation, and a purpose thereof is to provide a technology for supporting appropriate entries of medical interview information.

In response to the above issue, a medical system according to one aspect of the present disclosure includes: an order information recording unit that records information on an order for a patient; an arrival state recording unit that records an arrival state that indicates whether or not a patient has arrived, in association with an order for the patient; an accepting unit that accepts patient identification information entered into a portable terminal device; an order retrieval unit that retrieves an order related to the patient identification information accepted by the accepting unit; a decision processing unit that decides to display an entry screen for medical interview information when the arrival state associated with an order related to the patient identification information indicates that the patient has arrived; and a display processing unit that displays the entry screen for the medical interview information on the portable terminal device, in response to the decision made by the decision processing unit.

Optional combinations of the aforementioned constituting elements, and implementation of the present disclosure in the form of methods, apparatuses, systems, recording media, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 shows a configuration of a medical system according to an embodiment;

FIG. 2 shows some of recorded contents in a recording unit;

FIG. 3 is a functional block diagram of an information processing device;

FIG. 4 shows a login screen;

FIG. 5 shows a patient ID entry screen;

FIG. 6 is a flowchart of processing for displaying a medical interview information entry screen;

FIG. 7 shows an example of the medical interview information entry screen;

FIG. 8 shows an example of a message indicating that the medical interview information has been registered;

FIG. 9 shows an example of a message;

FIG. 10 shows a modification of the recorded contents in the recording unit;

FIG. 11 shows another modification of the recorded contents in the recording unit;

FIG. 12 shows an implementation information entry screen;

FIG. 13 shows a screen displaying a past examination report;

FIG. 14 shows a screen displaying endoscopic images captured in the past; and

FIG. 15 shows an entry screen for basic information regarding an examination.

### DETAILED DESCRIPTION

The disclosure will now be described by reference to the preferred embodiments. This does not intend to limit the scope of the present disclosure, but to exemplify the disclosure.

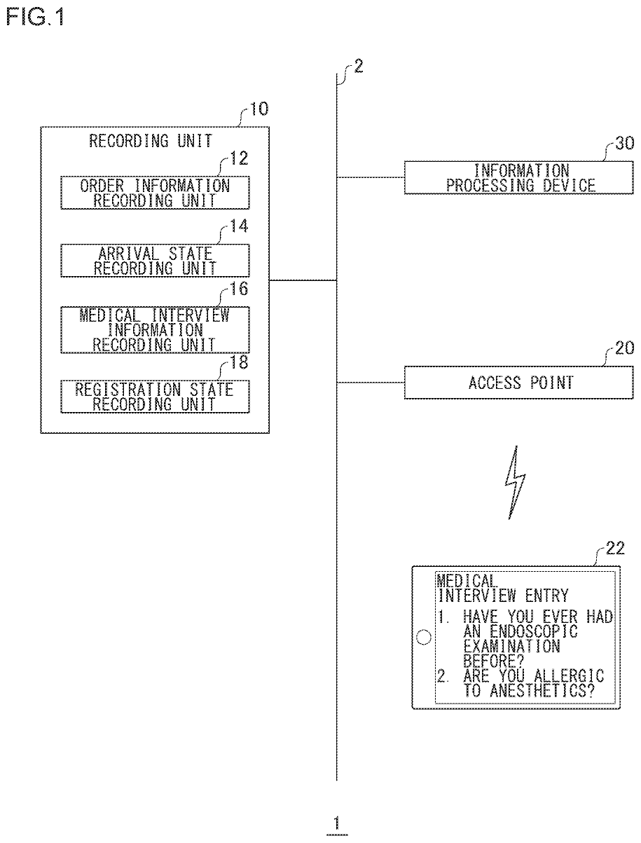

FIG. 1 shows a configuration of a medical system 1 according to an embodiment of the present disclosure. The medical system 1 of the embodiment is installed in a medical facility, such as a hospital, and includes a recording unit 10, an access point (hereinafter, also referred to as “AP”) 20, a portable terminal device 22, and an information processing device 30. The recording unit 10, the AP 20, and the information processing device 30 are connected by a network 2, such as a local area network (LAN), so as to communicate with each other. The portable terminal device 22 is connected to the recording unit 10 and the information processing device 30 via the AP 20 so as to communicate therewith.

The recording unit 10 includes an order information recording unit 12 that records information on an order for a patient, an arrival state recording unit 14 that records an arrival state regarding a patient's arrival at a facility, a medical interview information recording unit 16 that registers medical interview information entered by a patient into the portable terminal device 22, and a registration state recording unit 18 that records a registration state of a patient's medical interview information.

In a medical facility constituted by multiple departments, the medical system 1 may be a departmental system provided in one department. Although the embodiment describes an example in which the medical system 1 is a system provided in an endoscopy department, the medical system 1 may be a system provided in another department. The portable terminal device 22 is an electronic device owned and managed by the endoscopy department and may be an electronic device such as a tablet equipped with a display function and a wireless communication function.

In the medical system 1, when a patient scheduled to undergo an examination on the day has arrived at the reception counter of the endoscopy department and completed the reception, a nurse hands to the patient the portable terminal device 22 on which an entry screen for medical interview information is displayed. The patient enters the medical interview information by answering the questions displayed on the entry screen. After answering all the questions and operating the finish button on the screen, the patient returns the portable terminal device 22 to the reception counter. The medical interview information thus entered is transmitted from the portable terminal device 22 to the recording unit 10 and recorded in the medical interview information recording unit 16. The medical system 1 of the embodiment provides a mechanism for displaying a medical interview information entry screen (an entry screen for medical interview information) for a patient easily and certainly on the portable terminal device 22 before a nurse hands the portable terminal device 22 to the patient.

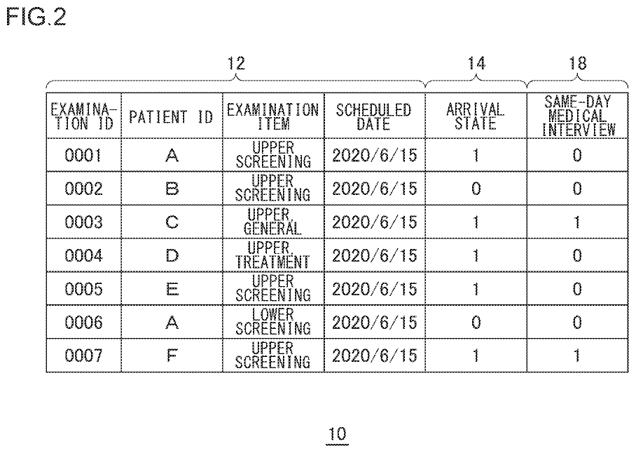

FIG. 2 shows some of the recorded contents in the recording unit 10. The order information recording unit 12 records information on an order for a patient. An endoscopic examination order is issued by an ordering system (not illustrated) and provided to the endoscopy department system. In this example, the order information recording unit 12 records an “examination ID” as identification information of an examination order, a “patient ID” as identification information of a patient, an “examination item” indicating the type of an endoscopic examination, and a “scheduled date” indicating the scheduled date of an examination. The order information recording unit 12 may further record information regarding the practitioner, the scheduled start time of the examination, and the examination room, for example.

The arrival state recording unit 14 records an arrival state, which indicates whether or not a patient has arrived at the endoscopy department, in association with an order for the patient. When a patient has arrived at the reception counter of the endoscopy department on the day of an examination, the patient completes the reception. For example, the patient shows his or her medical registration card (patient ID card) to a medical staff member at the reception counter, and the medical staff member enters the patient ID into the endoscopy department system, thereby completing the reception for the examination. When an automatic reception machine is provided in the endoscopy department system, the patient may also complete the reception for the examination by having the patient ID recorded on the medical registration card read by the automatic reception machine. Based on the patient ID thus accepted, the endoscopy department system registers, in the arrival state recording unit 14, arrival information indicating that the patient has arrived at the endoscopy department, in association with the order scheduled for the patient on the day.

Before the patient's arrival at the endoscopy department, the arrival state recording unit 14 has recorded nonarrival information indicating that the patient has not yet arrived. When the patient has completed the reception, the arrival state recording unit 14 records arrival information indicating that the patient has arrived. The arrival state may be represented by a flag value, and the arrival information indicating that the patient has arrived may be indicated by a flag value of 1, while the nonarrival information indicating that the patient has not yet arrived may be indicated by a flag value of 0.

In the example shown in FIG. 2, for a patient with the patient ID “A” (hereinafter, referred to as a “patient A”), two examination orders identified by the examination IDs of “0001” and “0006” are registered. In this way, when multiple examinations are scheduled for one patient on the same day, the endoscopy department system registers the arrival state for each examination in the arrival state recording unit 14. In the example shown in FIG. 2, the arrival information is recorded for the examination of upper screening (the examination ID “0002”) scheduled for the patient A, whereas the nonarrival information is recorded for the examination of lower screening (the examination ID “0006”) also scheduled for the patient A. This means that the patient A has completed the reception for the upper screening examination but has not completed the reception for the lower screening examination.

After completing the reception, the patient waits in a waiting room for his or her turn to be examined. Before the patient's examination starts, a nurse displays the medical interview information entry screen for the patient on the portable terminal device 22 and hands to the patient the portable terminal device 22 with the entry screen displayed. The portable terminal device 22 includes a touch panel, and the patient enters the medical interview information with touch operation on the screen or using a special stylus pen. Upon completion of the entry of all the medical interview information, the patient touches a “finish button” displayed on the touch panel. Accordingly, the portable terminal device 22 transmits the medical interview information thus entered to the recording unit 10. At the time, the medical interview information recording unit 16 registers the medical interview information in association with the order for the patient, and the registration state recording unit 18 records registration information indicating that the medical interview information has been registered, in association with the order for the patient.

The registration state recording unit 18 records a registration state, which indicates whether or not the medical interview information has been registered, in association with an order for a patient. The registration state of the medical interview information may be represented by a flag value, and the registration information indicating that the medical interview information has been registered may be indicated by a flag value of 1, while nonregistration information indicating that the medical interview information has not been registered may be indicated by a flag value of 0. In the embodiment, the registration of the medical interview information is performed on the premise that the arrival information has been registered. Therefore, the situation where the medical interview information is registered while the arrival information has not been registered does not occur in principle.

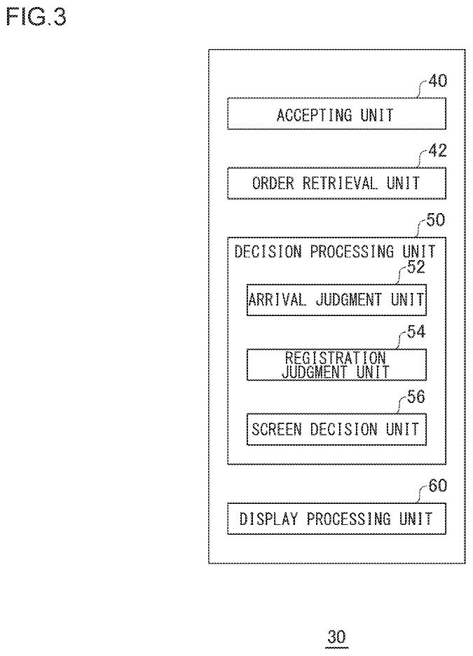

FIG. 3 shows functional blocks of the information processing device 30. The information processing device 30 is configured as a server device in the medical system 1 and includes an accepting unit 40, an order retrieval unit 42, a decision processing unit 50, and a display processing unit 60. The decision processing unit 50 includes an arrival judgment unit 52, a registration judgment unit 54, and a screen decision unit 56.

The configuration shown in FIG. 3 may be implemented by an arbitrary processor, memory, auxiliary storage device, and other LSIs in terms of hardware, and by a memory-loaded program or the like in terms of software. FIG. 3 illustrates functional blocks implemented by coordination thereof. Therefore, it will be understood by those skilled in the art that these functional blocks may be implemented in a variety of forms by hardware only, software only, or a combination thereof.

In the medical system 1, a nurse hands the portable terminal device 22 displaying the entry screen for medical interview information (medical interview information entry screen), to a patient waiting in a waiting room. Since the medical interview information entered by the patient on the entry screen needs to be registered as the patient's medical interview information in the medical interview information recording unit 16, the entry screen displayed on the portable terminal device 22 needs to be related to the patient to whom the portable terminal device 22 is handed. Accordingly, the medical system 1 implements a mechanism for allowing a nurse to easily and certainly display the entry screen for a patient to whom the portable terminal device 22 is handed.

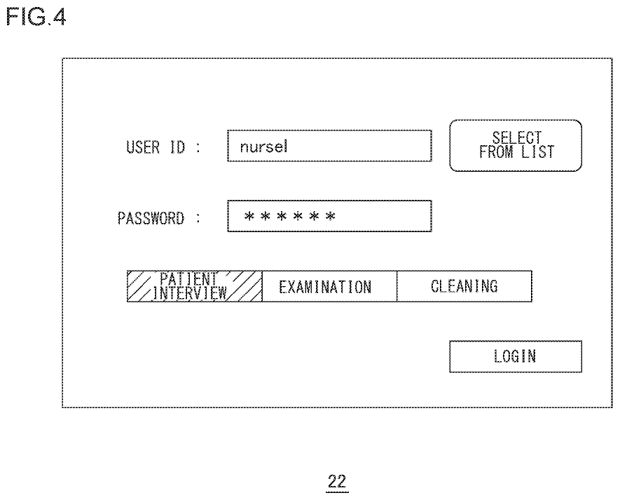

FIG. 4 shows a login screen displayed on the portable terminal device 22. On the login screen, a nurse enters his or her user ID and password. The login screen displays options for multiple functions of “patient interview”, “examination”, and “cleaning”. When the nurse selects the “patient interview” function and operates the “login” button, the user ID and password are transmitted to the endoscopy department system for authentication. When the user ID and password are successfully authenticated, the nurse with the user ID of “nurse1” logs in the “patient interview” function of the portable terminal device 22. By logging in the selected function, the nurse becomes able to use the function.

The portable terminal device 22 only allows execution of a function that the user logged in and does not execute functions that the user has not logged in. In this example, since the nurse has logged in the “patient interview” function, the portable terminal device 22 can perform only the functions associated with the display of the medical interview information entry screen (medical questionnaire) and does not perform any function for accessing examination information or cleaning information. This eliminates the possibility that the patient to whom the portable terminal device 22 has been handed may access the examination information or cleaning information through an erroneous operation.

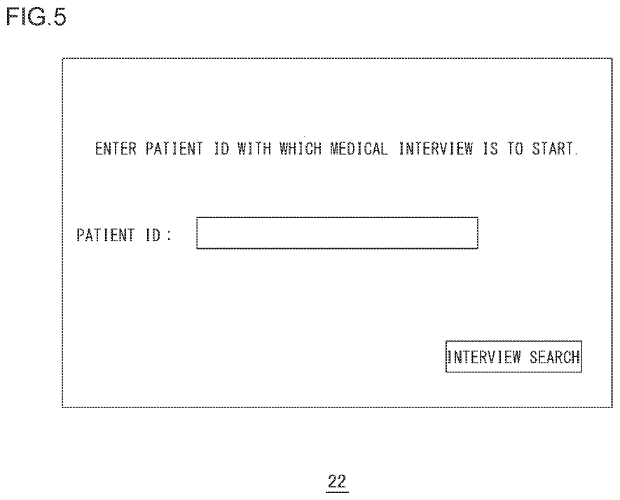

FIG. 5 shows an entry screen for patient ID (patient ID entry screen). When the nurse has logged in the “patient interview” function, the portable terminal device 22 displays a screen on which the patient ID is entered. When the nurse enters, on the patient ID entry screen, the patient ID of the patient with whom the interview is to start and operates the “interview search” button, the portable terminal device 22 transmits the patient ID thus entered to the information processing device 30. In order to enter the patient ID accurately without making a mistake, the nurse may enter the patient ID while looking at the patient's medical registration card (patient ID card) near the patient. If the portable terminal device 22 is provided with a patient ID reading function, the patient ID may be read directly from the patient's medical registration card so as to avoid incorrect entry of the patient ID.

The information processing device 30 accepts the patient ID entered into the portable terminal device 22. Based on the patient ID thus accepted, the information processing device 30 performs processing for displaying the medical interview information entry screen on the portable terminal device 22.

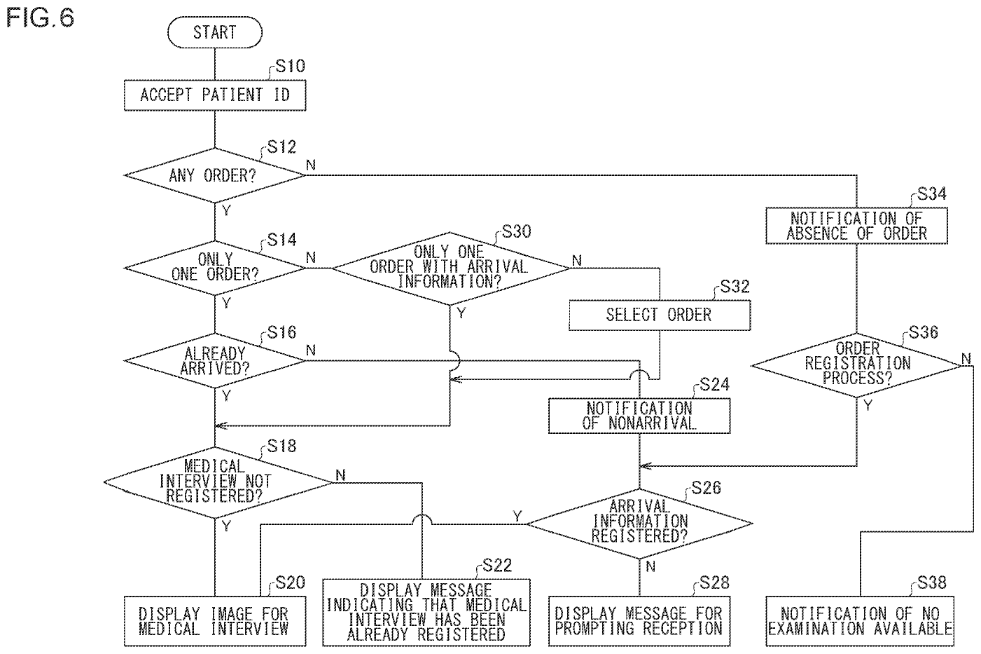

FIG. 6 is a flowchart of processing for displaying the medical interview information entry screen on the portable terminal device 22. When a patient ID entered into the portable terminal device 22 is transmitted to the information processing device 30, the accepting unit 40 accepts the patient ID (S10). The order retrieval unit 42 retrieves an order related to the patient ID accepted by the accepting unit 40, from the order information recording unit 12 (S12). More specifically, the order retrieval unit 42 retrieves an order related to the accepted patient ID, among examination orders of which the scheduled examination date is set to the date of the acceptance of the patient ID.

The order information recording unit 12 may record only information on an order of which the scheduled examination date is the current day. For example, the order information recording unit 12 may acquire and record information on the day's examination orders from the ordering system prior to the start of the endoscopy department's operations for the day. In this case, since only the information on the day's examination orders is recorded in the order information recording unit 12, the order retrieval unit 42 has only to retrieve, among the examination orders recorded in the order information recording unit 12, an examination order related to the patient ID entered into the portable terminal device 22.

If there is an examination order related to the accepted patient ID (Y at S12), the order retrieval unit 42 judges whether there is one examination order related to the accepted patient ID or there are multiple such examination orders (S14).

With reference to FIG. 2, when the accepting unit 40 accepts the patient ID “D”, for example, the order retrieval unit 42 retrieves an order related to the patient ID “D” and identifies the examination order with the examination ID “0004”. Hereinafter, the patient with the patient ID “X” may be referred to as the “patient X”, and the examination order with the examination ID “****” may be referred to as the “examination order ‘****’”. The order for the patient D is only the examination order “0004” (Y at S14), and hence, the one examination order “0004” is related to the patient D.

Meanwhile, when the accepting unit 40 accepts the patient ID “A”, the order retrieval unit 42 retrieves an order related to the patient ID “A” and identifies the examination order “0001” and the examination order “0006”. Thus, there are two orders, the examination order “0001” and the examination order “0006”, related to the patient A (N at S14), i.e., multiple examination orders are related to the patient A.

When there is only one order related to a patient (Y at S14), the arrival judgment unit 52 judges whether the arrival state associated with the order indicates that the patient has arrived (S16). Since the arrival state recording unit 14 records the arrival state of a patient in association with an order for the patient, the arrival judgment unit 52 refers to the arrival state associated with the order. For example, when the accepting unit 40 has accepted the patient ID “D”, since the arrival state with the flag value of 1 is associated with the examination order “0004” related to the patient D (see FIG. 2), the arrival judgment unit 52 judges that the patient D has arrived (Y at S16).

The registration judgment unit 54 then judges whether the registration state associated with the order related to the patient indicates that the medical interview information has not been registered (S18). Since the registration state recording unit 18 records the registration state, which indicates whether or not the medical interview information has been registered, in association with an order for a patient, the registration judgment unit 54 refers to the registration state associated with the order. With the examination order “0004” related to the patient D, the registration state with the flag value of 0 is associated (see FIG. 2), and therefore the registration judgment unit 54 judges that the medical interview information of the patient D has not been registered (Y at S18). At the time, the screen decision unit 56 decides to display the medical interview information entry screen on the portable terminal device 22 and, in response to the decision made by the screen decision unit 56, the display processing unit 60 displays the medical interview information entry screen on the portable terminal device 22 (S20).

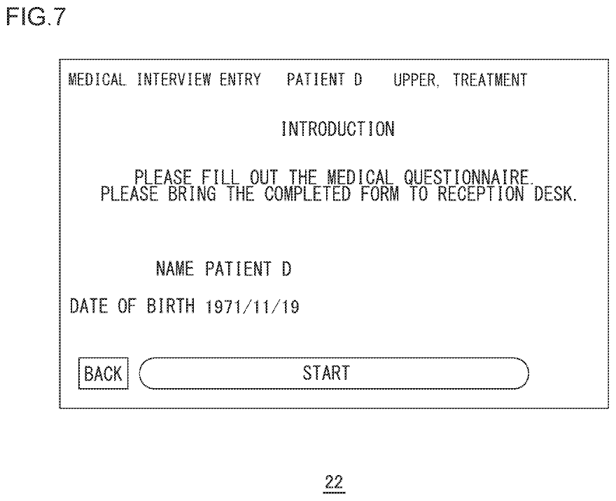

FIG. 7 shows an example of the medical interview information entry screen displayed on the portable terminal device 22. A nurse hands the portable terminal device 22 displaying the entry screen to the patient D. On the start screen, the name of the patient D is displayed; accordingly, the nurse confirms the name with the patient D before handing the portable terminal device 22 to the patient. Upon the operation of the “start” button by the patient D, the medical interview starts.

The data constituting the medical interview information entry screen may be transmitted from the display processing unit 60 of the information processing device 30 to the portable terminal device 22. Also, an application for displaying the entry screen appropriate for an examination item may be installed on the portable terminal device 22, and the application may display the entry screen on the portable terminal device 22. When the patient D has entered all the medical interview information and operates the “finish button”, the medical interview information thus entered is transmitted to the recording unit 10 and recorded in the medical interview information recording unit 16. Also, the registration state of the medical interview information associated with the examination order “0004” is updated to the flag value of 1.

According to the processing procedure of the embodiment, when a nurse merely logs in the “patient interview” function and enters the patient ID on the patient ID entry screen, the medical interview information entry screen is immediately displayed on the portable terminal device 22. In other words, after the patient ID entry screen is displayed, when the screen decision unit 56 decides to display the medical interview information entry screen, the display processing unit 60 immediately switches the patient ID entry screen to the medical interview information entry screen, without displaying any other screen between the screens.

Since the “patient interview” function of the embodiment does not include any function other than the functions associated with the display of the medical interview information entry screen, the situation can be prevented where a patient entering the medical interview information views, through an erroneous operation, the medical interview information of other patients or information regarding the nurse's work, for example. Also, once the nurse enters the patient ID, the information processing device 30 automatically judges if the conditions of S12, S14, S16, and S18 are met and decides the display of the medical interview information entry screen. Therefore, the medical interview information entry screen related to the patient can be displayed easily and certainly.

At S18, if the registration state indicating that the medical interview information has been registered is associated with the examination order (N at S18), the display processing unit 60 will display on the portable terminal device 22 a message indicating that the medical interview information has been registered (S22).

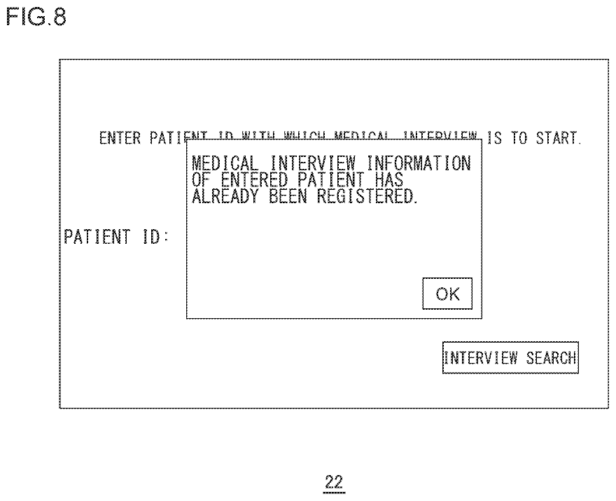

FIG. 8 shows an example of the message indicating that the medical interview information has been registered. Viewing the message, the nurse recognizes that the medical interview information has already been registered and that further registration of the medical interview information is unnecessary.

In the above procedure, the screen decision unit 56 decides to display the medical interview information entry screen on the portable terminal device 22 on the condition that the medical interview information has not been registered (Y at S18). However, the screen decision unit 56 may decide the display of the medical interview information entry screen regardless of the condition of S18.

At S16, if the arrival judgment unit 52 has judged that the arrival state associated with the order indicates that the patient has not arrived (N at S16), the display processing unit 60 will display on the portable terminal device 22 a message indicating that the patient has not yet arrived (S24). This situation occurs when the nurse enters, on the patient ID entry screen, the patient ID of a patient who has not completed the reception.

At the time, if the nurse is authorized to register the arrival information of the patient, i.e., to perform reception of the patient, the nurse may register the arrival information of the patient (Y at S26).

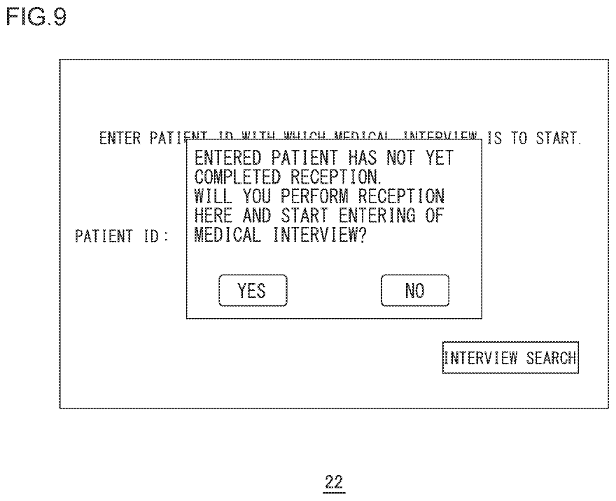

FIG. 9 shows an example of a message displayed for a nurse authorized to register the arrival information. When the arrival state of a patient indicates that the patient has not arrived, the display processing unit 60 displays a message indicating that the patient has not yet arrived (S24) and also displays a message inquiring whether or not to perform reception. When the nurse selects “YES”, the reception is performed, and the arrival information is registered in the arrival state recording unit 14 (Y at S26). When the arrival information is registered in the arrival state recording unit 14, the screen decision unit 56 decides to display the medical interview information entry screen on the portable terminal device 22, and the display processing unit 60 immediately displays the medical interview information entry screen on the portable terminal device 22 (S20). Meanwhile, when the nurse selects “NO” (N at S26), the reception is not performed, and a message prompting the reception process at the reception counter is displayed (S28).

At S14, when there are multiple examination orders related to the patient ID accepted at S10 (N at S14), the arrival judgment unit 52 judges whether the arrival information is associated with only one order (S30). For example, when the accepting unit 40 has accepted the patient ID “A”, the order retrieval unit 42 identifies the examination order “0001” and the examination order “0006” related to the patient A, and the arrival judgment unit 52 judges that the arrival information is associated with the examination order “0001” whereas the nonarrival information is associated with the examination order “0006”. Therefore, there is only one examination order with which the arrival information is associated (Y at S30), and, in this case, the registration judgment unit 54 judges that the medical interview information is not associated with the examination order “0001” (Y at S18). Accordingly, the screen decision unit 56 decides to display the medical interview information entry screen for the examination item (upper screening) of the examination order “0001”, and the display processing unit 60 displays the medical interview information entry screen on the portable terminal device 22 (S20). Thus, when there are multiple examination orders related to the patient ID accepted at S10, the screen decision unit 56 decides to display the medical interview information entry screen for the order for which the arrival state indicates that the patient has arrived, thereby enabling immediate display of the medical interview information entry screen.

Meanwhile, at S30, when there is more than one examination order with which the arrival information is associated (N at S30), the screen decision unit 56 decides to display a selection screen on which an examination order for which the medical interview information is to be entered is selected, and the display processing unit 60 displays an order selection screen on the portable terminal device 22. On the order selection screen, the nurse selects the order of an examination item for which the medical interview information is to be entered (S32). When the order is selected, on the condition that the medical interview information is not associated with the selected examination order (Y at S18), the display processing unit 60 displays the medical interview information entry screen on the portable terminal device 22 (S20).

At S12, if there is no order related to the patient ID accepted by the accepting unit 40 (N at S12), the display processing unit 60 will display on the portable terminal device 22 a message indicating that no order for the current day has been issued for the patient (S34). Accordingly, the nurse recognizes that there is no order.

At the time, the display processing unit 60 may display a screen for issuing an order on the portable terminal device 22. The nurse may hand the portable terminal device 22 to a healthcare professional (such as a doctor) who is authorized to issue orders, so as to have an order for the patient issued from the portable terminal device 22 (Y at S36). Thereafter, when the nurse registers the arrival information of the patient (Y at S26), the display processing unit 60 displays the medical interview information entry screen on the portable terminal device 22 (S20).

Meanwhile, if the order registration process cannot be carried out (N at S36), the display processing unit 60 will display on the portable terminal device 22 a message indicating that the examination cannot be conducted due to the absence of the order (S38).

Described above is an operation in which a patient, who has completed the reception, enters medical interview information into the portable terminal device 22 while waiting in a waiting room for his or her turn to be examined. If the patient is hospitalized in an inpatient ward of a medical facility, the patient may be able to enter the medical interview information in the inpatient ward before going to the endoscopy department for reception. Also, if the patient is an outpatient, the patient may be able to enter the medical interview information on the patient's smart phone or the like prior to the examination. By entering the medical interview information prior to the reception, the patient can undergo the examination promptly after the patient arrives at the endoscopy department and completes the reception.

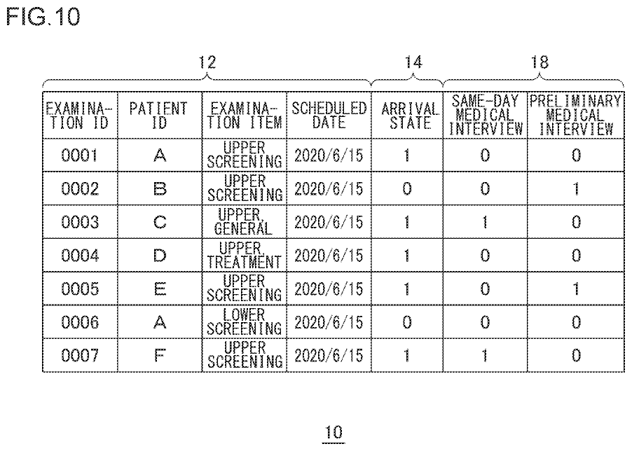

FIG. 10 shows a modification of the recorded contents in the recording unit 10. Compared to the recorded contents shown in FIG. 2, the recording unit 10 shown in FIG. 10 records information indicating an implementation state of a “preliminary medical interview” conducted prior to the reception.

In the modification, the registration state recording unit 18 records a registration state that indicates whether or not same-day medical interview information, which is entered after the reception, has been registered and also records a registration state that indicates whether or not preliminary medical interview information, which is entered before the reception, has been registered, in association with an order for a patient. Each of the registration states of the same-day medical interview information and the preliminary medical interview information may be represented by a flag value, and registration information indicating that the medical interview information has been registered may be indicated by a flag value of 1, while nonregistration information indicating that the medical interview information has not been registered may be indicated by a flag value of 0.

In the processing procedure shown in FIG. 6, the registration judgment unit 54 judges whether the registration state associated with an order indicates that the medical interview information has not been registered (S18). In the modification, since the registration state recording unit 18 records the registration state of the same-day medical interview information and the registration state of the preliminary medical interview information in association with an order for the patient, the registration judgment unit 54 refers to the registration states. At the time, if the registration judgment unit 54 judges that either the same-day medical interview information or the preliminary medical interview information has been registered (N at S18), the display processing unit 60 may display on the portable terminal device 22 a message indicating that medical interview information has been registered (S22). If only the preliminary medical interview information has been registered, the display processing unit 60 may display on the portable terminal device 22 a message for asking whether to conduct the same-day medical interview.

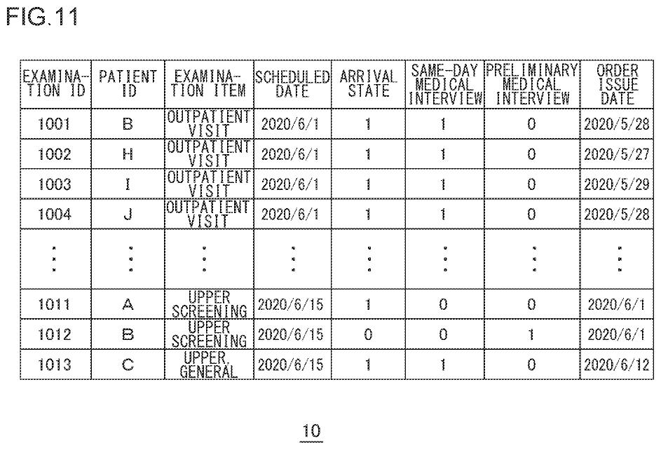

FIG. 11 shows another modification of the recorded contents in the recording unit 10. Compared to the recorded contents shown in FIG. 10, the recording unit 10 shown in FIG. 11 records an “order issue date” on which an order was issued.

An examination is often scheduled as a result of an outpatient visit. In this case, the order for the examination is issued on the day of the outpatient visit. Accordingly, the registration judgment unit 54 judges whether the order issue date of an examination related to a patient coincides with the date of a past outpatient visit of the same patient. In the example shown in FIG. 11, the order issue date of an upper screening examination for the patient B (2020/6/1) coincides with the date of a past outpatient visit of the patient B (2020/6/1). Therefore, the registration judgment unit 54 may relate the medical interview information entered by the patient B at the outpatient visit, to the upper screening examination for the patient B.

At the time, the display processing unit 60 may display the medical interview information entered at the outpatient visit on the portable terminal device 22. The nurse may refer to the past medical interview information to check items related to the current examination before displaying a medical interview screen for the examination.

The present disclosure has been described with reference to an embodiment. The embodiment is intended to be illustrative only, and it will be obvious to those skilled in the art that various modifications to a combination of constituting elements or processes could be developed and that such modifications also fall within the scope of the present disclosure. Some or all of the configurations described as the configurations of the information processing device 30 may be provided in the portable terminal device 22. In the embodiment, the recording unit 10 and the information processing device 30 installed in a medical facility are described. However, the functions of the recording unit 10 and/or the information processing device 30 may be implemented by a cloud server connected to the Internet. Also, although the embodiment describes the case where the medical interview information is associated with an order for an endoscopic examination, the order is not limited to an examination order.

On the login screen shown in FIG. 4, when a nurse enters his or her user ID and password, selects the “examination” function, and operates the “login” button, the user ID and password are transmitted to the endoscopy department system for authentication. When the user ID and password are successfully authenticated, the nurse with the user ID of “nurse1” logs in the “examination” function of the portable terminal device 22.

In the following, there will be described examples of screens provided to a nurse who has logged in the “examination” function.

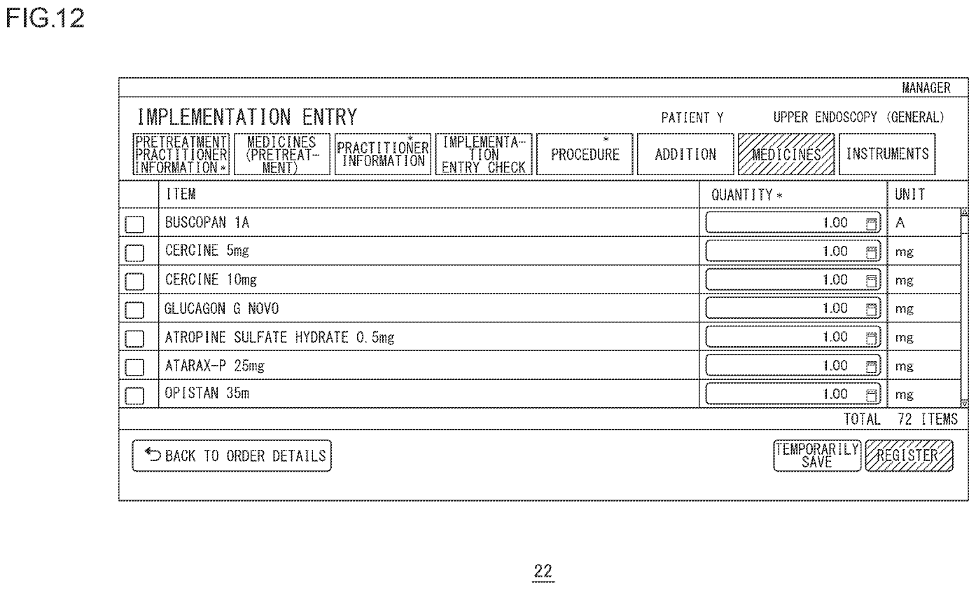

FIG. 12 shows an entry screen for implementation information (an implementation information entry screen). A nurse enters, on the entry screen, implementation information, such as medicines and instruments used in the pretreatment and examination. Since it is used for calculation of medical service fees, the implementation information needs to be entered accurately. With the implementation information entry screen provided by the portable terminal device 22, the nurse can promptly and certainly enter the implementation information at the time when a medicine or an instrument is used.

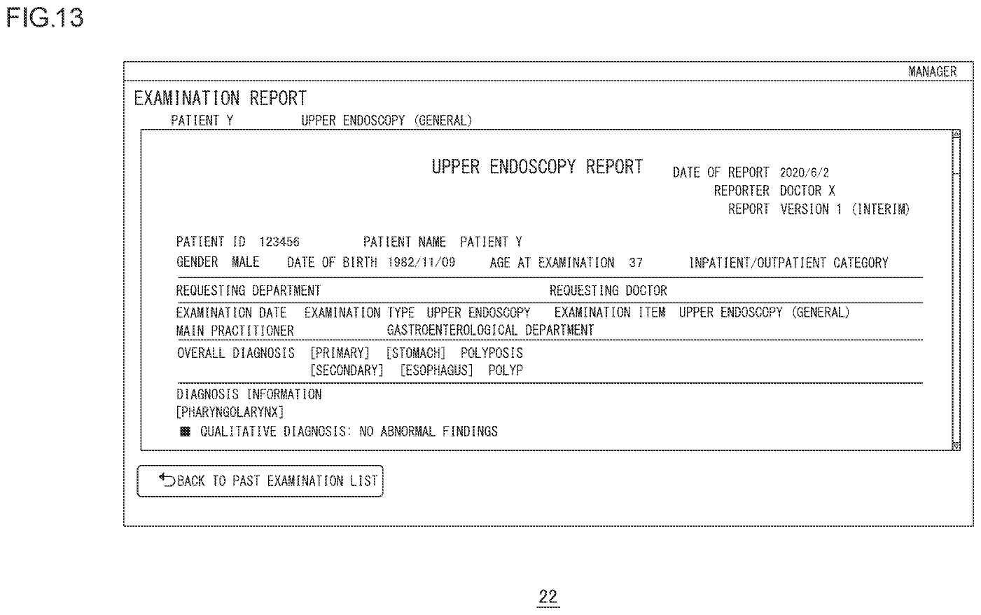

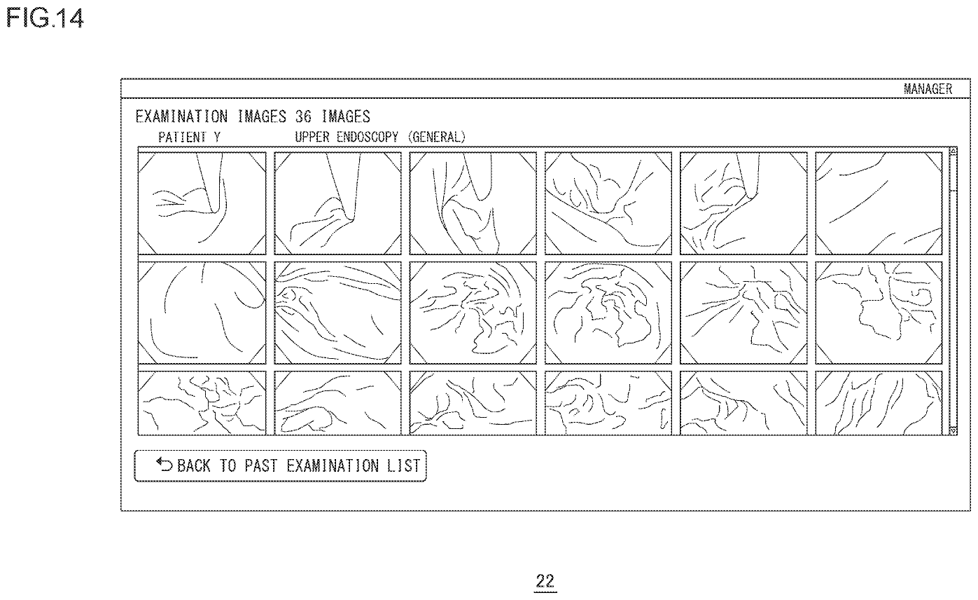

FIG. 13 shows a screen displaying a past examination report, and FIG. 14 shows a screen displaying endoscopic images captured in the past. The nurse who has logged in the “examination” function can view past examination reports and endoscopic images of the patient. With a past examination result reference screen provided by the portable terminal device 22, the nurse can access the past examination information regarding the patient.

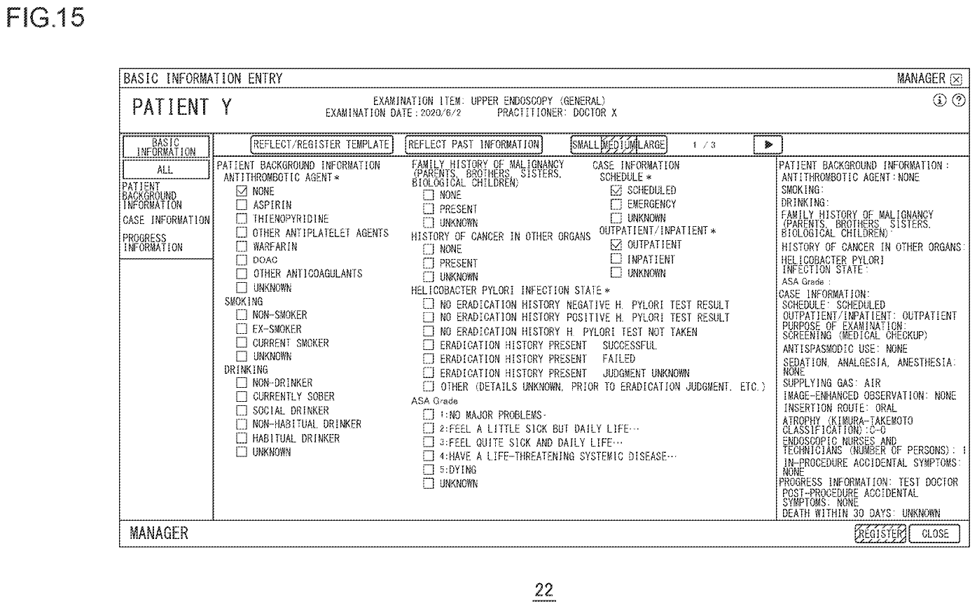

FIG. 15 shows an entry screen for basic information regarding an examination. By enabling entry of the basic information on a single screen, the time and effort required to enter the basic information can be reduced.

What is claimed is:

1. An information processing device comprising a processor including hardware, wherein the processor is configured to:
accept patient identification information entered into a portable terminal device;
retrieve an examination order related to the patient identification information that has been accepted;
refer to an arrival state associated with examination identification information of the examination order, the arrival state indicating whether or not a patient has arrived;
decide to display an entry screen for medical interview information when the arrival state indicates that the patient has arrived; and
display on the portable terminal device the entry screen for the medical interview information corresponding to the examination identification information.

2. The information processing device according to claim 1, wherein the processor is configured to:
judge whether there is one examination order or there are a plurality of the examination orders related to the patient identification information; and
display, when there are the plurality of the examination orders related to the patient identification information and when only the arrival state associated with the examination identification information of one examination order among the plurality of the examination orders indicates that the patient has arrived, the entry screen for the medical interview information corresponding to the examination identification information of the one examination order on the portable terminal device.

3. The information processing device according to claim 1, wherein the processor is configured to:
switch, when displaying the entry screen for the medical interview information is decided after an entry screen for the patient identification information is displayed, the entry screen for the patient identification information to the entry screen for the medical interview information.

4. The information processing device according to claim 1, wherein the processor is configured to:
decide to display the entry screen for the medical interview information when the medical interview information has not been registered; and
display the entry screen for the medical interview information corresponding to the examination identification information.

5. The information processing device according to claim 1, wherein, the processor is configured to:
decide to display, when a plurality of the examination orders related to the patient identification information are identified and when there is more than one examination order with which the arrival state indicating that the patient has arrived is associated, a selection screen on which a plurality of the examination orders are selected.

6. An entry screen display method for displaying an entry screen for medical interview information, the entry screen display method comprising:
accepting patient identification information entered into a portable terminal device;
retrieving an examination order related to the patient identification information that has been accepted;
referring to an arrival state associated with examination identification information of the examination order, the arrival state indicating whether or not a patient has arrived;
deciding to display an entry screen for medical interview information when the arrival state indicates that the patient has arrived; and
displaying on the portable terminal device the entry screen for the medical interview information corresponding to the examination identification information.

7. The entry screen display method according to claim 6, comprising:
judging whether there is one examination order or there are a plurality of the examination orders related to the patient identification information, and
displaying, when there are a plurality of the examination orders related to the patient identification information and when only the arrival state associated with the examination identification information of one examination order among the plurality of the examination orders indicates that the patient has arrived, the entry screen for the medical interview information corresponding to the examination identification information of the one examination order on the portable terminal device.

8. The entry screen display method according to claim 6, comprising:
switching, when displaying the entry screen for the medical interview information is decided after an entry screen for the patient identification information is displayed, the entry screen for the patient identification information to the entry screen for the medical interview information.

9. The entry screen display method according to claim 6, comprising:
deciding to display the entry screen for the medical interview information when the medical interview information has not been registered, and
displaying the entry screen for the medical interview information corresponding to the examination identification information.

10. The entry screen display method according to claim 6, comprising:
deciding to display, when a plurality of the examination orders related to the patient identification information are identified and when there is more than one examination order with which the arrival state indicating that the patient has arrived is associated, a selection screen on which a plurality of the examination orders are selected.

11. A medical system comprising a storage device and a processor including hardware, the storage device records information on an examination order for a patient, and an arrival state that indicates whether or not a patient has arrived in association with examination identification information of an examination order for the patient,
the processor is configured to:
accept patient identification information entered into a portable terminal device;
retrieve the examination order related to the patient identification information that has been accepted;
decide to display an entry screen for medical interview information when the arrival state associated with the examination order related to the patient identification information indicates that the patient has arrived; and
display on the portable terminal device the entry screen for the medical interview information corresponding to the examination identification information.

12. The medical system according to claim 11, wherein the processor is configured to:
judge whether there is one examination order or there are a plurality of the examination orders related to the patient identification information; and
display, when there are a plurality of the examination orders related to the patient identification information and when only the arrival state associated with the examination identification information of one examination order among the plurality of the examination orders indicates that the patient has arrived, the entry screen for the medical interview information corresponding to the examination identification information of the one examination order on the portable terminal device.

13. The medical system according to claim 11, wherein the processor is configured to:
switch, when displaying the entry screen for the medical interview information is decided after an entry screen for the patient identification information is displayed, the entry screen for the patient identification information to the entry screen for the medical interview information.

14. The medical system according to claim 11, wherein the storage device records a registration state that indicates whether or not the medical interview information has been registered, in association with the examination identification information of the examination order for the patient, and
the processor is configured to:
decide to display the entry screen for the medical interview information when the registration state associated with the examination order indicates that the medical interview information has not been registered; and
display the entry screen for the medical interview information corresponding to the examination identification information.

15. The medical system according to claim 11, wherein the processor is configured to:
decide to display, when a plurality of the examination orders related to the patient identification information are identified and when there is more than one examination order with which the arrival state indicating that the patient has arrived is associated, a selection screen on which a plurality of the examination orders are selected.

16. The medical system according to claim 14, wherein the processor is configured to:
display, when the registration state associated with the examination order indicates that the medical interview information has been registered, a message indicating that the medical interview information has been registered.

17. The medical system according to claim 11, wherein the processor is configured to:
display, when the examination order related to the patient identification information that has been accepted does not exist, a message indicating that no examination order for the current day has been issued for the patient.
